(19) United States
(12) Patent Application Publication
(10) Pub. No.: US 2002/0192262 A1
(43) Pub. Date: Dec. 19, 2002

(54) 1'-Acetoxychavicol acetate for tuberculosis treatment

(75) Inventors: Prasit Palittapongarnpim, Bangkok (TH); Chalermpol Kirdmanee, Bangkok (TH); Prasat Kittakoop, Prathumthani (TH); Kamolchanok Rukseree, Bangkok (TH)

Correspondence Address: Mr. Prasit Palittapongarnpim, 113 Thailand Science Park, Paholyothin Rd., Klong 1, Klong Luang, Pathumthani, 12120, TH

(73) Assignee: National Science and Technology Development Agency, Klong 1, Klong Luang, Prathumthani (TH)

(21) Appl. No.: 10/152,570

(22) Filed: May 23, 2002

(30) Foreign Application Priority Data
Jun. 18, 2001 (TH) 066318

(51) Int. Cl. A61F 13/00

(52) U.S. Cl. 424/422

(57) ABSTRACT

1′-Acetoxychavicol acetate is a compound not known before to possess anti-tuberculous activity. The above data revealed that the compound was active against the standard H37Ra strain as well as several clinical isolates at the concentration well below the toxic concentration against various mammalian cells. The compound is therefore potentially useful as an therapeutic and preventive agent for tuberculosis as well as an antiseptic agent against the bacteria.

### CROSS-REFERENCE TO RELATED APPLICATION

Not Applicable

### STATEMENT REGARDING FEDERALLY SPONSORED RESEARCH OR DEVELOPMENT

Not Applicable

### REFERENCE TO SEQUENCE LISTING

Not Applicable

### BACKGROUND OF THE INVENTION

1′-Acetoxychavicol acetate, whose structure is shown below, is a natural compound, which is found in some plants in the family Zingiberaceae especially in greater galingale (Alpinia galanga (Linn.) Sw.) and big galingale (Alpinia nigra (Gaertn.) B. L. Burtt). It is not found in several of other members of this family, such as Zingiber officinale, Kaempferia galanga and Alpinia officinarum, which is used as medicine in China. Galingales have been used as herb and food in Thailand and other countries in Asia for a long time.

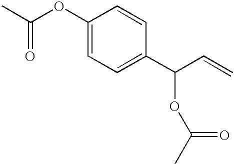

Many investigators reported growth-inhibiting activities of 1-acetoxychavicol acetate against many organisms. It could inhibit the growth of various fungi (Jassen, A. M. and Scheffer, J. J. C. 1985), including many dermatophytic fungi such as Trichophyton mentagrophytes, Trichophyton rubrum, Trichophyton concentricum and Epidermophyton floccosum with the minimal inhibitory concentrations (MIC) between 50-250 μg/ml. It also inhibited the growth of several other fungi such as Rhizopus stolonifer, Penicillium expansum, Aspergilus niger, albeit with higher MIC. This compound could not inhibit the growth of the yeast Candida albicans, and many bacteria, such as Escherichia coli, Pseudomonas aeruginosa and Bacillus subtilis but could slightly inhibit the growth of Staphylococcus aureus.

There has been no existing report on the inhibitory activity against the growth of M. tuberculosis and other mycobacterium of this compound.

1′-Acetoxychavicol acetate can inhibit the formation of many tumor and cancer in mice experimental models, such as skin cancer (Murakami, A. et.al., 1996), bile duct cancer (Miyauchi, M. et.al. 2000), esophageal cancer (Kawabata, K. et.al. 2000), large intestinal cancer (Tanaka, T. et.al. 1997 and Tanaka, T. et.al. 1997), oral cancer (Ohnishi, M. et.al. 1996) and liver tumor (Kobayashi, Y. et.al. 1998).

The mechanisms of action of the compound were not clear. The compound could inhibit the activation of tumor virus such as Ebstein-Barr virus (Marukami, A. et.al. 2000 and Kondo, A. et.al. 1993), and could inhibit the function of xanthine oxidase and NADPH oxidase (Noro, T. et.al. 1998 and Tanaka, T. et.al. 1997). These enzymes involve in superoxide anion production, which is one of the spontaneously occurring toxic substances in the body (Nakamura, Y. et.al. 1998 and Murakami, A. et.al. 1996).

1′-Acetoxychavicol acetate can inhibit nitric oxide synthase production in RAW264 (mice macrophage) cell line when stimulated with mice interferon-γ or bacterial lipopolysaccharides (Ohata, T. et.al. 1998). 1′-acetoxychavicol acetate at the concentration of 250 could completely inhibit nitric oxide synthase production when stimulated with 100 ng/ml of bacterial lipopolysaccharide. The enzyme production was 80% inhibited when stimulated with 100 ng/ml of interferon-γ. This compound was about 10 times more potent than the other nitric oxide synthase inhibitors such as curcumin, nonsteroidal anti-inflammatory drugs, genistein and ω-3 polyunsaturated fatty acids. 1′-Acetoxychavicol could inhibit nitric oxide synthase by inhibiting the destruction of IκB-α protein, which is an inhibitor of NF-κB (a transcription factor), leading to the decrease of the NF-κB activity and, consequently, resulting in decreased nitric oxide synthase production. It also inhibited other transcription factors such as AP-1 and Stat-1. It has been suggested that nitric oxide, which is produced by nitric oxide synthase, involves in tumor formation.

Greater galingale (Alpinia galanga (Linn.) Sw. or Languas galanga (Linn) Stuntz.) and big galingale (Alpinia nigra (Gaertn.) B.L. Burtt) belong to the family Zingiberaceae. The galingales are found in Asia, from India, Indonesia to Philippines. They are used as food and herb in Thailand. As herb, the galingales are generally used as anti-flatulence, to decrease the gastric discomfort and to treat dermatophytic fungal infection. It was noted in a Thai ethnomedicinal textbook that galingale oil could be used for tuberculosis treatment.

It was reported that greater galingale did not produce acute toxic effects in mice even at the dose as high as 3 g/kg body weight and did not have chronic toxicity when given to mice at the dose of 100 mg/kg bodyweight for 90 days. It was found that it did not affect the body weight or the weights of any organs including heart, lung, liver, spleen, and kidney. It increased the number of red blood cells but not white blood cells. It increased the weight of sex organs in male mice with the increase of sperm number and sperm movement. It was not toxic to sperm (Qureshi, S. et.al. 1992 and Mokkhasmit, M. et.al. 1971). In contrast, it decreased the toxicity of cyclophosphamide in mice (Qureshi, S. et.al. 1994).

1′-Acetoxychavicol acetate can be found in high concentration, of about 1.5%-2.8% of dry weight, in the greater galingale root (De Pooter, H. L. et.al. 1985), but less in the leaf (Jassen, A. M. and Scheffer, J. J. C. 1985). The configuration of 1′-acetoxychavecol naturally found in the galingale is in S-form.

Many chemicals have been reported in greater galingale. These included galingin, 3-methygalangin (Ramachandran, N. and Gunasegaran, R. 1982), 1′-hydroxychavicol acetate, 1′-acetoxyeuginol acetate (Jassen, A. M. and Scheffer, J. J. C. 1985), p-hydroxycinnamaldehyde, [di-(p-hydroxy-cis-styryl)] methane (Barik, B. R. 1987), galanal A, galanal B, galanolactone, (E)-8(17),12-labddiene-15,16-dial, (E)-8β(17), epoxylabd-12-ene-15,16-dial (Morita, H. and Itokawa, H. 1987).

Tuberculosis, caused by Mycobacterium tuberculosis, is an important communicable disease. Mycobacterium is a genus of bacteria, which have special cell membrane structures different from other bacteria. This renders most antibiotics unable to enter the bacterial cells, leading to failure in inhibiting the growth of the bacteria. Tuberculosis, therefore, requires special drugs for treatment.

Anti-tuberculosis drugs can be divided into two groups. The first line drugs, are highly effective and of relatively low toxicity. The second line drugs, are less effective and/or of relatively high toxicity. The drugs are used when the bacteria resist the first line drugs.

There are 5 first line drugs, which are isoniazid, rifampin, pyrazinamide, ethambutol and streptomycin. Standard tuberculosis treatment requires 4 in the 5 drugs. There must be isoniazid and rifampin with two other drugs, usually pyrazinamide and ethambutol or streptomycin. The 6-month-long treatment starts with these 4 drugs for 2 months, followed by treatment with isoniazid and rifampin for 4 months. This is because only isoniazid and rifampin are highly effective in killing the bacteria. When M. tuberculosis resists to any of pyrazinamide, ethambutol or streptomycin, the treatment requires the switch to second line drugs and still might be able to complete the treatment in 6 months. On the other hand, if the organisms resist to isoniazid or rifampin, even the switch to other effective drugs may not render the treatment being successful in 6 months. The treatment may need to be lengthened up to 18 months especially if the organisms resist rifampin. The M. tuberculosis is, therefore, called multi-drug resistant when resists to both isoniazid and rifampin. Multi-drug resistant tuberculosis is a very serious public health problem because it can not be cured in 6 months or the worst, not at all. This is due to the fact that the bacteria may become gradually resisting other drugs during the treatment. The patients may have no serious symptoms even though the treatment can not eliminate the bacteria because the drugs may control the organisms to some extent. The patients can therefore survive and transmit the resistant strains to the other people.

The presence of limited number of the highly effective drugs is a major problem in tuberculosis control. Although, isoniazid and rifampin have been discovered for 30 years, there have been limited efforts to identify new highly effective drugs.

### DETAILED DESCRIPTION OF THE INVENTION

The discovery and development of new anti-tuberculous drugs are usually started by showing that a new compound can inhibit the growth of M. tuberculosis in vitro. The method includes culturing the bacteria in artificial medium, which contains the compound and then observing the growth of the bacteria. The compounds with higher activity will inhibit the growth of M. tuberculosis at a lower concentration than the compounds with lower activity. The activity of each drug can be compared by its minimal inhibitory concentration (MIC).

The growth of M. tuberculosis can be measured by several methods such as observing colony formation in solid media or turbidity in liquid media. However, the observation of the slow growing M. tuberculosis is possible only after a long period of incubation. Many investigators tried to find a way to observe the growth in a shorter time. The M. tuberculosis usually grows more rapidly in liquid media than in solid media. Therefore, the tests for drug development are usually done in liquid media.

Several indirect growth observation methods have been developed for clinical use. These include observing the production of radioactive carbon dioxide in BACTEC460 system (Middlebrook, G. et.al. 1997), the oxygen in Mycobacterium Growth Indicator Tube (Pfyffer, G. E. et.al. 1997) or the bioluminescence from the luciferase enzyme that is transducted into M. tuberculosis by a specially-engineered virus (Arain, T. M. et.al.). Most of these methods can decrease the test period from 3-4 weeks to only 7-10 days.

Many of the systems, marketed for clinical use, require high amount of media and consequently high amount of samples. They are, therefore, not suitable for drug development. The methods specifically designed for drug development are usually done in microplate. The small wells allow the use of small amount of culture media and tested compounds. A popular microplate test uses the bacteria containing luciferase enzyme as surrogate host. The growing bacteria produce the luciferase enzyme, rendering it bioluminescent. Another method measures the oxygen content in the microplate by observing the color change of Alamar Blue (Collins, L. and Franzblau, S. G. 1997) or other dyes.

Anti-tuberculous drugs must have low toxicity because the patients need to ingest it for a long time. Primary testing for toxicity is usually done by incubates the candidate compounds with human cells which the cells were cultivated in vitro and then observes the cytopathic effects. In principle, every chemical compound is toxic to human cells at a high enough concentration. The chemical compound that may be used as drug must have the ability to inhibit growth of organisms at a lower concentration and is toxic to human cells at a higher concentration, such as at the concentration more than 10 fold higher than the MIC. The compound can then theoretically be administered to human to achieve concentration between MIC and the toxic concentration.

The appropriate compounds for the 1′-acetoxychavicol acetate may be readily prepared by methods known to those skilled in the art. The preferred method for the preparation of 1′-acetoxychavicol acetate involves the following steps a) to d):

a) Preparation of 1′-acetoxychavicol Acetate from Galingale

Extraction and purification of the compound was done starting from slicing the root of greater galingale (Alpinia galanga (Linn.) Sw.) or big galingale (Alpinia nigra (Gaertn) B. L. Burtt). The slices were air-dried and then ground, following by dichloromethane extraction. The extracts were then dried, resolubilized and purified by silica gel column. After elution with dicloromethane:hexane (1:1), the elute was distilled at 170-190° C. to recover pure 1′-acetoxychavicol acetate. The yield of 1′acetoxychavicol acetate was about 60 gm/kg of the galingales.

b) Preparation of Bacteria to Test 1′-acetoxychavicol Acetate Against M. tuberculosis

Mycobacterium tuberculosis H₃₇Ra strain (ATCC 25166) was grown in 100 ml of Middlebrook 7H9 broth supplemented with 0.2% glycerol, 1.0 gm/L of casitone, 10% OADC, and 0.05% Tween 80. The complete medium was referred to as 7H9GC-Tween. The bacteria were incubated in 500-ml flasks on a rotary shaker at 200 rpm and 37° C. until the optical density at 550 nm reached 0.4-0.5. The bacteria were washed twice with phosphate-buffered saline and then suspended in 20 ml of phosphate-buffered saline. The suspension was passed through an 8-μm-pore-size filter to eliminate clumps. The number of the bacteria in the filtrates was counted by plating the bacteria in Middlebrook 7H10 agar. The filtrates were stored at −80° C.

c) Microplate Alamar Blue assays (MABA)

Anti-tuberculous testing was performed in a 96-well microplate as previously described (Collins, L. and Franzblau, S. G. 1997). Outer perimeter wells were filled with sterile water to prevent dehydration of the test wells. Crude extracts were initially diluted in dimethyl sulfoxide, and then were diluted to a concentration of 400 μg/ml in Middlebrook 7H9 medium containing 0.2% V/V glycerol and 1.0 gm/L casitone (7H9GC). The wells in rows B to G in columns 2, 4, 5, 6, 8, 9, 10 of the microplate were inoculated with 100 μl of 7H9GC. The wells in column 11 were inoculated with 200 μl of the medium to serve as media controls (M). Bacteria (only) controls (B) were set-up in column 10. One hundred microliters of each crude extract solution (400 μg/ml) were added to three wells in one row in columns 2 (or 6), 3 (or 7) and 4 (or 8). One hundred microliters was transferred from column 4 (or 8) to column 5 (or 9), the contents of the wells in column 5 (or 9) were mixed well and then 100 μl of mixed medium were discarded. The wells in columns 2 and 6 served as test sample controls.

Frozen bacterial inocula were diluted 1:200 in 7H9GC medium. One hundred microliters of the bacteria were added to the wells in rows B to G in columns 3 (or 7), 4 (or 8), 5 (or 9) and 10 resulting in final bacterial titers of about 5×10⁴ CFU/ml. The wells in column 10 served bacteria (only) controls (B). Final concentrations of extracts were 200, 100 and 50 μg/ml in columns 3 (or 7), 4 (or 8) and 5 (or 9), respectively.

The plates were sealed with Parafilm and were incubated at 37° C. for 5 days. At day 6 of incubation, 20 μl of Alamar Blue reagent and 12.5 μl of 20% Tween 80 were added to well B10 (B) and B11 (M). The plates were re-incubated at 37° C. for 24 h. Wells were observed at 24 h for color change from blue to pink. If the B wells became pink by 24 h, reagent was added to the entire plate. If the well remained blue, the additional M and B wells was tested daily until a color change occurred at which time reagents were added to all remaining wells. The microplates were resealed with Parafilm and were then incubated at 37° C. The results were recorded at 24 h post-reagent addition.

A blue color in the well was interpreted as no growth, reflecting the activity of the test compound in the well. A pink color was scored as growth and reflected the lack of activity of the test compound. A few wells appeared violet after 24 h of incubation, but they invariably changed to pink after another day of incubation and thus were scored as growth (while the adjacent blue wells remained blue).

When 1′-acetoxychavicol acetate was found to be active at the concentration of 50 μg/ml, the activity of the compound was tested in the second plate containing the compound at two-fold serially diluted from 50 to 0.025 μg/ml. 1′-acetoxychavicol acetate can inhibit the growth of M. tuberculosis at the concentration of 0.1 μg/ml or higher but not at the concentration of 0.05 μg/ml or lower. The MIC of 1′-acetoxychavicol acetate against M. tuberculosis H₃₇Ra was therefore 0.1 μg/ml.

The activity of the compound was also tested for 30 clinical strains of M. tuberculosis isolated from patients in Thailand. The MICs were found to be between 0.1-0.5 μg/ml. The clinical isolates included isoniazid and/or rifampin resistant strains.

d) The Toxicity of 1′-acetoxychavicol Acetate

1′-acetoxychavicol acetate was tested for toxicity by incubating it with Vero cells (African green monkey kidney cell line from American Type Culture Collection USA). 1′-acetoxychavicol acetate was dissolved with dimethyl sulfoxide and then diluted in the culture medium of the Vero cells (Eagle's minimum essential with 10% heat-inactivated fetal bovine serum and antibiotics). The Vero cells and the compound were incubated together in a 96-well microplate at the cell concentration of 1.9×10⁴ cells/ 190 μl/well, in a CO₂ incubator at 37° C. for 3 days. The numbers of the cells in the wells were then determined by a staining method (Skehan, P. 1990). The cells were firstly fixed by 50% cold trichloroacetic acid (TCA) at 4° C. for 30 minutes. The cells were then washed with water 4 times. After drying, the cells were stained with 0.05% sulforhodamine B in 1% acetic acid for 30 minutes, washed with 1% acetic acid 4 times and dried at room temperature. Finally, 10 mM Tris-base pH10 was added. The absorbance at 510 nm of test wells was measured by an ELISA microplate reader. The absorbance was proportionate to the number of the viable cells in the wells. The toxic level of the compound was recorded as the concentration that rendered the number of viable cells being less than half of the negative control wells, which contained the cells with DMSO but not the compound. The test was done at least 3 times per concentration. Ellipticine was used as positive control. The toxic level of 1′-acetoxychavicol acetate against Vero cells was found to be 2.0 μg/ml, which was 20 times higher than the MIC against M. tuberculosis H₃₇Ra.

1-Acetoxychavicol acetate was also tested for toxicity against three other mammalian cell lines, namely L929 (mouse lung cells), BHK21 (hamster kidney cells) and HepG2 (human liver cells) by culturing the cells in microplates together with various concentration of the compounds. The toxic levels were again defined as the concentration that decrease the viability of the cells by half compared to the negative control, which contain no compound. The viability of these cells were determined by adding MTT (3-[4,5-dimethylthiazol-2-yl]-2,5-diphenyltetrazolium bromide) solution into wells after 48 hours of co-incubation of the cells with the compound. The viable cells converted the soluble MTT to insoluble formazan precipitate. After 4 hours of incubation, aqueous phase of the wells was removed and dimethyl sulfoxide was added to disslove the formazan. Sorensen's glycine buffer pH 10.5 was then added and the absorbance at 570 nm was measured and compared to the absorbance of the negative control wells.

The toxic levels of the compound for L929 and BHK21 cells were found to be 7.0-8.5 μg/ml, while the toxic level against HepG2 cells was 23.4 μg/ml.

### REFERENCE

1. Arain, T. M., Resconi, A. E., Hickey, M. J, and Stover, C. K. Bioluminescence screening in vitro (Bio-Siv) assays for high-volume antimycobacterial drug discovery. Antimicrob Agents Chemother. 1996;40:1536-41.

2. Barik, B. R., Kundu, A. B, and Dey, A. K. Two phenolic constituents from Alpinia galanga rhizome. Phytochemistry 1987;26:2126-2127.

3. Collins, L., and Franzblau, S. G. Microplate alamar blue assay versus BACTEC 460 system for high- throughput screening of compounds against Mycobacterium tuberculosis and Mycobacterium avium. Antimicrobl Agents Chemother 1997;41:1004-1009.

4. De Pooter, H. L., Omar, M. N., Coolsaet, B. A., and Schamp, N. M. The essential oil of greater galanga (Alpinia galanga) from Malaysia. Phytochemistry 1985;24:93-96.

5. Jassen, A. M. and Scheffer, J. J. C. Acetoxyhydroxychavicol acetate, an antifungal component of Alpinia galanga. Planta Med 1985;6:507-11.

6. Kawabata, K., Tanaka, T., Yamamoto, T. et. al. Suppression of N-nitrosomethylbenzylamine-induced rat esophageal tumorigenesis by dietary feeding of 1′-acetoxychavicol acetate. Jpn J. Cancer Res 2000;91:148-155.

7. Kobayashi, Y., Nakae, D., Akai, H. et. al. Prevention by 1′-acetoxychavicol acetate of the induction but not growth of putative preneoplastic, glutathione-S-transferase placental form-positive, focal lesions in the livers of rats fed a choline-deficient, L-amino acid-defined diet. Carcinogenesis 1998;19:1809-1814.

8. Kondo, A., Ohigashi, H., Murakami, A., Suratwadee, J., and Koshimizu, K. 1′-Acetoxychavicol acetate as a potent inhibitor of tumor-promoter-induced Epstein-Barr virus activation from Languas galanga, a traditional Thai condiment. Biosci Biotechnol Biochem 1993;57:1344-1345.

9. Marukami, A., Toyota, K., Ohura, S., Koshimizu, K., and Ohigashi, H. Structure-activity relationships of (1′S)-1′-acetoxychavicol acetate, a major constituent of a Southeast Asian condiment plant Languas galanga, on the inhibition of tumor-promoter-induced Epstein-Barr virus activation. J. Agric Food Chem 2000;48:1518-1523.

10. Middlebrook, G., Reggiardo, Z., and Tigertt, W.D. Automatable radiometric detection of growth of Mycobacterium tuberculosis in selective media. Am Rev Respir Dis 1977; 115: 1067-1069.

11. Mitsui, S., Kobayashi, S., Nagahori, H., and Ogiso, A. Constituents from seeds of Alpinia galanga Wild and their anti-ulcer activities. Chem Pharm Bull 1976;24:2377-2382.

12. Miyauchi, M., Nishikawa, A., Furukawa, F. et. al. Inhibitory effects of 1′acetoxychavicol acetate on N-nitrosobis (2-oxopropyl) amine-induced initiation of cholangiocarcinogenesis in syrian hamsters. Jpn J. Cancer Res 2000;91:477-481.

13.Mokkhasmit, M., Sawasdimongkol, K., and Satravaha, P. Toxicity study of Thai medicinal plants. Bull Dept Med Sci, Thailand 1971; 12, 2-4: 36-66.

14. Morita, H. and Itokawa, H. Cytotoxic and antifungal diterpenes from the seed of Alpinia galanga. Planta Med 1988;9:117-120.

15. Murakami, A., Ohura, S., Nakamura, Y. et. al. 1′acetoxychavicol acetate, a superoxide generator inhibitor, potently inhibits tumor promotion by 12-O-tetradecanoylphorbol-13-acetate in ICR mouse skin. Oncology 1996;53:386-391.

16.Nakamura, Y., Marukami, A., Ohto, Y., Torikai, K., Tanaka, T., and Ohigashi, H. Suppression of tumor promoter induced oxidative stress and inflammatory responses in mouse skin by a superoxide generator inhibitor 1′-acetoxychavicol acetate. Cancer Res 1998;58:4832-4839.

17. Noro, T., Sekiya, T., Katoh, M. et. al. Inhibitors of xanthine oxidase from Alpinia galanga. Chem Pharm Bull 1988;36:244-248.

18. Ohata, T., Fukudal, K., Murakami, A., Ohigashi, H., Sugimural, T., and Wakabashil, K. Inhibition by 1′-acetoxychavicol acetate of lipopolysaccharide- and interferon-gamma-induced nitric oxide production through suppression of inducible nitric oxide synthase gene expression in RAW264 cells. Carcinogenesis 1998;19:1007-1012.

1. Use of 1-Acetoxychavicol acetate as treatment agent for tuberculosis.

2. Use of 1-Acetoxychavicol acetate as defined in claim 1 alone or use as one of drug combination in any treatment agent for tuberculosis.

3. Use of 1-Acetoxychavicol acetate as treatment agent as defined in claim 2 for tuberculosis in human.

4. Use of 1-Acetoxychavicol acetate as treatment agent as defined in claim 2 for tuberculosis in animal.

5. Use of 1-Acetoxychavicol acetate as preventive agent for the development of tuberculous diseases in human.

6. The use of 1-Acetoxychavicol acetate in claim 5 wherein the tuberculous diseases in human are infected by M. tuberculosis.

7. Use of 1-Acetoxychavicol acetate as preventive agent for the development of tuberculous diseases in animals.

8. The use of 1-Acetoxychavicol acetate in claim 7 wherein the tuberculous diseases in animals are infected by M. tuberculosis.

9. Use of 1-Acetoxychavicol acetate as defined in claim 5 alone or use as one of drug combination in any preventive agent for the development of tuberculous diseases in human.

10. The use of 1-Acetoxychavicol acetate in claim 9 wherein the tuberculous diseases in human are infected by M. tuberculosis.

11. Use of 1 -Acetoxychavicol acetate as defined in claim 5 alone or use as one of drug combination in any preventive agent for the development of tuberculous diseases in animals.

12. The use of claim 11 wherein the tuberculous diseases in animals are infected by M. tuberculosis.

13. Use of 1-Acetoxychavicol acetate as disinfecting agent for contamination of M. tuberculosis in inanimate objects.

14. The use of 1-Acetoxychavicol acetate in claim 13 wherein the inanimate object is medical equipment.

15. The use of 1-Acetoxychavicol acetate in claim 13 wherein the inanimate object is environment.

16. Use of 1-Acetoxychavicol acetate as defined in claim 13 alone or use as one of drug combination in any preventive agent for contamination of M. tuberculosis in inanimate objects.
